(19) United States
(12) Patent Application Publication
(10) Pub. No.: US 2005/0203310 A1
(43) Pub. Date: Sep. 15, 2005

(54) Process for the production of aliphatic carboxylic acid esters

(75) Inventors: Kyoichi Watanabe, Oita-shi (JP); Hiroshi Uchida, Oita-shi (JP)

Correspondence Address: SUGHRUE MION, PLLC, 2100 PENNSYLVANIA AVENUE, N.W., SUITE 800, WASHINGTON, DC, 20037, US

(73) Assignee: SHOWA DENKO K.K., Minato-ku, Tokyo 105-8518 (JP)

(21) Appl. No.: 10/343,486

(22) PCT Filed: Nov. 8, 2002
(86) PCT No.: PCT/JP02/11686

§ 371 Date: May 27, 2003

Related U.S. Application Data
(60) Provisional application No. 60/389,281, filed on Jun. 18, 2002.

(30) Foreign Application Priority Data
Jun. 13, 2002 (JP) 2002-172502

(51) Int. Cl. C07C 67/04

(52) U.S. Cl. 560/241

(57) ABSTRACT

In a process for producing a lower aliphatic carboxylic acid ester by esterifying a lower aliphatic carboxylic acid and a lower olefin into a lower aliphatic carboxylic acid ester using an acid catalyst in a vapor phase, when the system is controlled to contain substantially no acetylene compounds, the deterioration of the catalyst can be remarkably prevented from proceeding and in turn a stable operation can be continuously performed for a long time.

### CROSS-REFERENCE TO RELATED APPLICATION

This application is an application filed under 35 U.S.C. §111(a) and claiming benefit pursuant to 35 U.S.C. §119(e)(1) of the filing date of the Provisional Application 60/389,281 filed Jun. 18, 2002, pursuant to 35 §111(b).

### TECHNICAL FIELD

The present invention relates to a process for producing a lower aliphatic carboxylic acid ester by reacting a lower olefin and a lower aliphatic carboxylic acid and also relates to a lower aliphatic carboxylic acid ester obtained by the production process.

### BACKGROUND ART

As is well known, a corresponding lower aliphatic carboxylic acid ester can be obtained by reacting a lower olefin and a lower aliphatic carboxylic acid in the presence of an acid catalyst. It is also known that in this reaction, a heteropolyacid and/or a heteropolyacid salt effectively acts as a catalyst. Specific examples of these conventional techniques include those described, for example, in Japanese Unexamined Patent Publications No. 4-139148 (JP-A-4-139148), No.4-139149 (JP-A-4-139149), No. 5-65248 (JP-A-5-65248), No. 5-163200 (JP-A-5-163200), No. 5-170699 (JP-A-5-170699), No. 5-255185 (JP-A-5-255185), No. 5-294894 (JP-A-5-294894), No. 6-72951 (JP-A-6-72951) and No. 9-118647 (JP-A-9-118647). Thus, development of catalysts having high initial activities is proceeding.

However, in the industrial production process, impurities derived from starting materials or by-products produced during the reaction give rise to deterioration of the catalyst and, in turn, problems such as reduction in the reaction result. Particularly, the catalyst deteriorates due to the effect of impurities contained in the starting materials on use of starting materials having a low purity or various impurities or by-products accumulated in the system on continuously performing a reaction by the process having a circulation system. This causes, for example, a vicious circle of further accelerating the side reaction.

### DISCLOSURE OF INVENTION

An object of the present invention is to provide a process for producing a lower aliphatic carboxylic acid ester by esterifying a lower aliphatic carboxylic acid with a lower olefin in a vapor phase, where the operation can be continuously and stably performed.

More specifically, the object of the present invention is to provide a process for producing a lower aliphatic carboxylic acid ester by esterifying a lower aliphatic carboxylic acid with a lower olefin in a vapor phase, where the impurities derived from starting materials or the compounds derived from by-products produced in the process having a circulation system are reduced to a low concentration based on the starting materials to thereby prevent, particularly, the deterioration of catalyst and to enable a continuous and stable operation for a long period of time.

The present inventors have made extensive studies to find a process for producing a lower aliphatic carboxylic acid ester by reacting a lower olefin and a lower aliphatic carboxylic acid, where deterioration of the catalyst hardly occurs and the operation can be continuously and stably performed for a long period of time.

As a result, it has been found that, in the process for producing a lower aliphatic carboxylic acid ester by esterifying a lower aliphatic carboxylic acid and a lower olefin into a lower aliphatic carboxylic acid ester using an acid catalyst in a vapor phase, when the system is controlled to contain substantially no acetylene compounds, the deterioration of the catalyst can be remarkably prevented from proceeding and in turn a stable operation can be continuously performed for a long time.

That is, the present invention (I) provides a process for producing a lower aliphatic carboxylic acid ester from a lower aliphatic carboxylic acid and a lower olefin in the presence of an acid catalyst, wherein the starting materials contain substantially no acetylene compounds.

The present invention (II) provides a lower aliphatic carboxylic acid ester produced by the process of the present invention (I).

### BRIEF DESCRIPTION OF THE DRAWINGS

The figures each is a schematic view showing the process according to one embodiment for carrying out the present invention.

FIG. 1 is a view showing a one-path process having no circulation step.

FIG. 2 is a view showing a process having a circulation step from a post step.

### BEST MODE FOR CARRYING OUT THE INVENTION

The present invention is described in detail below.

The term “acetylene compound” as used herein refers to a lower olefin having a carbon-carbon triple bond. Specific examples thereof include acetylene, methyl acetylene and vinyl acetylene. The “acetylene compound” more preferably means acetylene.

In particular, polymerization of an acetylene compound, which can occur under the esterification reaction conditions for producing a lower aliphatic carboxylic acid ester, may be a problem. However, the problems are not limited thereto.

In the production process of a lower aliphatic carboxylic acid ester of the present invention, the concentration of acetylene compounds in the starting materials is controlled to 25 ppm or less in terms of the molar ratio to the total of the acetylene compounds and the lower olefin, and this is effective for reducing the deterioration rate of catalyst and in turn for continuously performing a stable operation for a long period of time.

The term “the concentration of acetylene compounds in the starting materials” as used herein means the concentration immediately before the inlet of a reactor for performing the esterification for producing a lower aliphatic carboxylic acid ester.

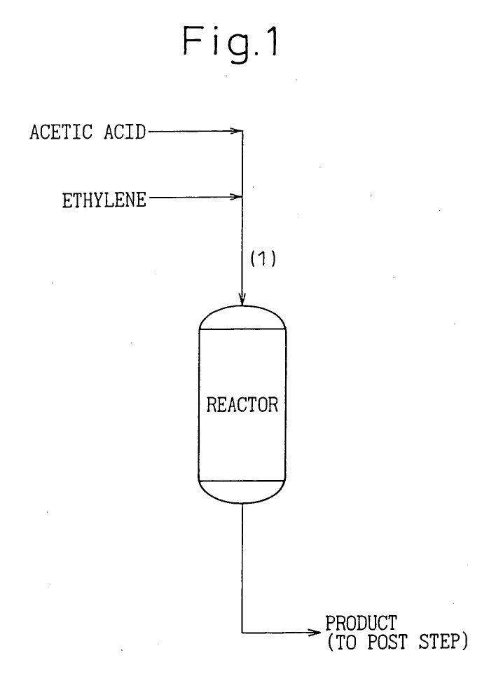

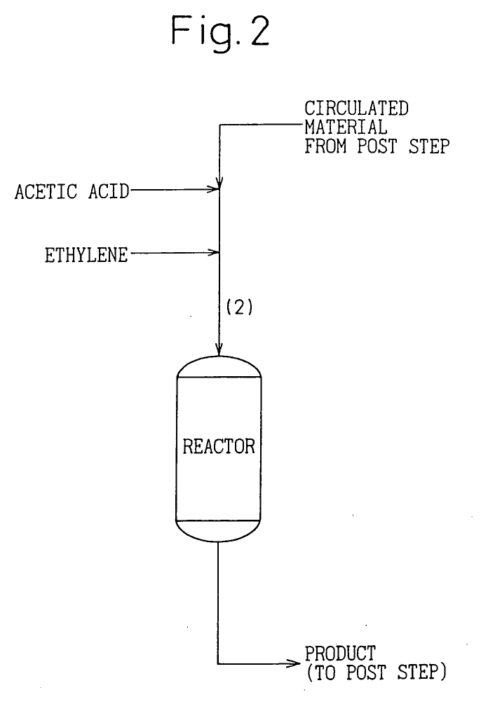

Specifically, for example, in the case where the reaction is performed in the one-path process having no circulation step as shown in FIG. 1, the concentration of acetylene compounds in the starting materials indicates the concentration immediately before the reactor inlet shown by (1). In the process having a circulation step from a post step as shown in FIG. 2, the concentration of acetylene compounds in the starting materials indicates the concentration immediately before the reactor inlet shown by (2). Of course, the present invention is not limited to these exemplified processes.

Accordingly, the term “starting materials” as used herein include, in addition to newly fed lower olefin and lower aliphatic carboxylic acid, unreacted starting materials after the reaction in a reactor, which are recovered through a post step, purified, if desired, and then fed to the reactor via a circulation system.

The position (1) in the process shown by FIG. 1 and the position (2) in the process shown by FIG. 2 are each generally kept at a temperature equal to the reaction temperature in the reactor. Accordingly, in the measurement of concentration at such a position, the sampling must be particularly designed. For example, the following method may be used. A part of a gas is sampled and cooled, the entire amount of the condensate collected is recovered and analyzed by gas chromatography, the effluent gas remaining uncondensed is measured on the flow rate of the gas flowing out within the sampling time and a part of the gas is sampled and analyzed by gas chromatography.

In the present invention, the starting materials preferably contain substantially no acetylene compounds. In particular, if the concentration of acetylene compounds exceeds 25 ppm in terms of the molar ratio to the total of the acetylene compounds and the lower olefin, the catalytic activity decreases at an extremely high rate and the catalyst life is very short. This is considered to occur because the acetylenes react on the catalyst to polymerize and thereby produce cokes and the active sites of the catalyst are covered by the cokes and, as a result, the catalyst is deactivated.

Accordingly, the concentration of acetylene compounds in the starting materials is preferably as low as possible and is preferably 10 ppm or less, more preferably 1 ppm or less. The “1 ppm or less” as used herein refers to the detection limit value in the acetylene analysis described, for example, in the present specification. It is preferred that acetylenes are substantially not detected.

The method for controlling the concentration of acetylene compounds in the starting materials to 25 ppm or less in terms of the molar ratio to the total of the acetylene compounds and the lower olefin is not particularly limited. Commonly known separation techniques may be used.

For example, fundamentally, the lower olefin used as a starting material is of course refined to reduce the contents of these compounds as much as possible. Also, a method of previously hydrogenating the acetylene compounds contained in the starting material by a known hydrogenation reaction to convert the acetylene compounds into alkenes or alkanes which do not inhibit the reaction is effective. The hydrogenation reaction is described, for example, in Japanese Unexamined Patent Publications No. 54-90101 (JP-A-54-90101), No. 55-87727 (JP-A-55-87727) and No. 59-59634 (JP-A-59-59634).

The acetylene compounds produced by the side reaction within the reaction system, which are a problem when a circulation system is employed, can be separated from the lower olefin by a method of allowing an appropriate solvent to absorb the main products (exclusive of a lower olefin), the starting materials and the by-products in the reaction gas flowing out from the reactor. Also, the starting material gas may be separated from the lower olefin by high-pressure or low-temperature distillation or by using a separation membrane or the like. Other than these specific examples, any method may be used as long as it is a method capable of controlling the concentration of acetylene compounds circulated and introduced into the reactor to 25 ppm or less in terms of the molar ratio to the total of the acetylenes and the lower olefin.

The lower aliphatic carboxylic acid as a starting material in the reaction of the present invention is preferably a lower aliphatic carboxylic acid having from 1 to 4 carbon atoms, more preferably a formic acid, an acetic acid, an acrylic acid, a propionic acid or a methacrylic acid, still more preferably an acetic acid or an acrylic acid. Of course, these may be used as a mixture of two or more thereof.

Examples of the lower olefin as a starting material in the reaction of the present invention include ethylene, propylene, n-butene, isobutene and a mixture of two or more thereof.

Examples of the acid catalyst which can be used in the present invention include compounds widely known in general as an acid catalyst, such as a heteropolyacid and a salt thereof, an ion-exchange resin, a mineral acid, zeolite and a composite metal oxide. Among these, a heteropolyacid and a heteropolyacid salt are preferred.

The heteropolyacid as used herein is a compound consisting of a center element and peripheral elements to which oxygen is bonded. The center element is usually silicon or phosphorus but may comprise any one atom selected from various atoms belonging to Groups 1 to 17 of the periodic table of elements. Specific examples thereof include cupric ion; divalent beryllium, zinc, cobalt and nickel ions; trivalent boron, aluminum, gallium, iron, cerium, arsenic, antimony, phosphorus, bismuth, chromium and rhodium ions; tetravalent silicon, germanium, tin, titanium, zirconium, vanadium, sulfur, tellurium, manganese, nickel, platinum, thorium, hafnium, cerium ions and other rare earth ions; pentavalent phosphorus, arsenic, vanadium and antimony ions; hexavalent tellurium ion; and heptavalent iodide ion, however, the present invention is not limited thereto. Specific examples of the peripheral element include tungsten, molybdenum, vanadium, niobium and tantalum, however, the present invention is not limited thereto.

These heteropolyacids are known also as a “polyoxoanion”, a “polyoxometallic salt” or a “metal oxide cluster”. Some structures of well-known anions are named after a researchers in this field, for example, Keggin, Wells-Dawson and Anderson-Evans-Perloff structure. These are described in detail in Poly-san no Kagaku, Kikan Kagaku Sosetsu (Chemistry of Polyacids, the Introduction of Chemistry Quarterly), No. 20, compiled by Nippon Kagaku Kai (1993). The heteropolyacid usually has a high molecular weight, for example, a molecular weight of 700 to 8,500, and includes not only a monomer but also a dimeric complex.

The heteropolyacid salt is not particularly limited as long as it is a metal salt or onium salt resulting from substituting a part or all of the hydrogen atoms of the heteropolyacid.

Specific examples thereof include metal salts such as those of lithium, sodium, potassium, cesium, magnesium, barium, copper, gold and gallium, and onium salts such as those of ammonia, however, the present invention is not limited thereto.

Particularly when the heteropolyacid is a free acid or a certain salt, the heteropolyacid exhibits a relatively high solubility in a polar solvent such as water or other oxygenated solvents. The solubility can be controlled by selecting an appropriate counter ion.

Preferred examples of the heteropolyacid which can be used as the catalyst in the present invention include:
silicotungstic acid H₄[SiW₁₂O₄₀]·xH₂O
phosphotungstic acid H₃[PW₁₂O₄₀]·xH₂O
phosphomolybdic acid H₃[PMo₁₂O₄₀]·xH₂O
silicomolybdic acid H₄[SiMo₁₂O₄₀]·xH₂O
silicovanadotungstic acid H₄₊ₙ[SiVₙW₁₂₋ₙO₄₀]·xH₂O
phosphovanadotungstic acid H₃₊ₙ[PVₙW₁₂₋ₙO₄₀]·xH₂O
phosphovanadomolybdic acid H₃₊ₙ[PVₙMo₁₂₋ₙO₄₀]·xH₂O
silicovanadomolybdic acid H₄₊ₙ[SiVₙMo₁₂₋ₙO₄₀]·xH₂O
silicomolybdotungstic acid H₄[SiMoₙW₁₂₋ₙO₄₀]·xH₂O
phosphomolybdotungstic acid H₃[PMoₙW₁₂₋ₙO₄₀]·xH₂O
   wherein n is an integer of 1 to 11 and x is an integer of 1 or more, however, the present invention is not limited thereto.

Among these, preferred are silicotungstic acid, phosphotungstic acid, phosphomolybdic acid, silicomolybdic acid, silicovanadotungstic acid and phosphovanadotungstic acid, more preferred are silicotungstic acid, phosphotungstic acid, silicovanadotungstic acid and phosphovanadotungstic acid.

The method for synthesizing these heteropolyacids is not particularly limited and any method may be used. For example, the heteropolyacid can be obtained by heating an acidic aqueous solution (pH: approximately from 1 to 2) containing a salt of molybdic acid or tungstic acid and a simple oxygen acid of heteroatom or a salt thereof. For isolating the heteropolyacid compound from the resulting aqueous heteropolyacid solution, a method of crystallizing and separating the compound as a metal salt may be used. Specific examples thereof are described in Shin Jikken Kagaku Koza 8, Muki Kagobutsuno Gosei (III) (New Experimental Chemistry Course 8, Synthesis (III) of Inorganic Compounds), 3rd ed., compiled by Nippon Kagaku Kai, issued by Maruzen, page 1413 (Aug. 20, 1984), however, the present invention is not limited thereto. The Keggin structure of the heteropolyacid synthesized can be identified by the X-ray diffraction or UV or IR measurement, in addition to the chemical analysis.

Particularly preferred examples of the heteropolyacid salt include a lithium salt, a sodium salt, a potassium salt, a cesium salt, a magnesium salt, a barium salt, a copper salt, a gold salt, a gallium salt and an ammonium salt of the above-described preferred heteropolyacids. Among these, more preferred are a lithium salt of silicotungstic acid and a cesium salt of phosphotungstic acid.

Specific examples of the heteropolyacid salt include a lithium salt of silicotungstic acid, a sodium salt of silicotungstic acid, a copper salt of silicotungstic acid, a gold salt of silicotungstic acid, a gallium salt of silicotungstic acid, a lithium salt of phosphotungstic acid, a sodium salt of phosphotungstic acid, a copper salt of phosphotungstic acid, a gold salt of phosphotungstic acid, a gallium salt of phosphotungstic acid, a lithium salt of phosphomolybdic acid, a sodium salt of phosphomolybdic acid, a copper salt of phosphomolybdic acid, a gold salt of phosphomolybdic acid, a gallium salt of phosphomolybdic acid, a lithium salt of silicomolybdic acid, a sodium salt of silicomolybdic acid, a copper salt of silicomolybdic acid, a gold salt of silicomolybdic acid, a gallium salt of silicomolybdic acid, a lithium salt of silicovanadotungstic acid, a sodium salt of silicovanadotungstic acid, a copper salt of silicovanadotungstic acid, a gold salt of silicovanadotungstic acid, a gallium salt of silicovanadotungstic acid, a lithium salt of phosphovanadotungstic acid, a sodium salt of phosphovanadotungstic acid, a copper salt of phosphovanadotungstic acid, a gold salt of phosphovanadotungstic acid, a gallium salt of phosphovanadotungstic acid, a lithium salt of phosphovanadomolybdic acid, a sodium salt of phosphovanadomolybdic acid, a copper salt of phosphovanadomolybdic acid, a gold salt of phosphovanadomolybdic acid, a gallium salt of phosphovanadomolybdic acid, a lithium salt of silicovanadomolybdic acid, a sodium salt of silicovanadomolybdic acid, a copper salt of silicovanadomolybdic acid, a gold salt of silicovanadomolybdic acid and a gallium salt of silicovanadomolybdic acid.

Among these, preferred are a lithium salt of silicotungstic acid, a sodium salt of silicotungstic acid, a copper salt of silicotungstic acid, a gold salt of silicotungstic acid, a gallium salt of silicotungstic acid, a lithium salt of phosphotungstic acid, a sodium salt of phosphotungstic acid, a copper salt of phosphotungstic acid, a gold salt of phosphotungstic acid, a gallium salt of phosphotungstic acid, a lithium salt of phosphomolybdic acid, a sodium salt of phosphomolybdic acid, a copper salt of phosphomolybdic acid, a gold salt of phosphomolybdic acid, a gallium salt of phosphomolybdic acid, a lithium salt of silicomolybdic acid, a sodium salt of silicomolybdic acid, a copper salt of silicomolybdic acid, a gold salt of silicomolybdic acid, a gallium salt of silicomolybdic acid, a lithium salt of silicovanadotungstic acid, a sodium salt of silicovanadotungstic acid, a copper salt of silicovanadotungstic acid, a gold salt of silicovanadotungstic acid, a gallium salt of silicovanadotungstic acid, a lithium salt of phosphovanadotungstic acid, a sodium salt of phosphovanadotungstic acid, a copper salt of phosphovanadotungstic acid, a gold salt of phosphovanadotungstic acid and a gallium salt of phosphovanadotungstic acid.

More preferred are a lithium salt of silicotungstic acid, a sodium salt of silicotungstic acid, a copper salt of silicotungstic acid, a gold salt of silicotungstic acid, a gallium salt of silicotungstic acid, a lithium salt of phosphotungstic acid, a sodium salt of phosphotungstic acid, a copper salt of phosphotungstic acid, a gold salt of phosphotungstic acid, a gallium salt of phosphotungstic acid, a lithium salt of silicovanadotungstic acid, a sodium salt of silicovanadotungstic acid, a copper salt of silicovanadotungstic acid, a gold salt of silicovanadotungstic acid, a gallium salt of silicovanadotungstic acid, a lithium salt of phosphovanadotungstic acid, a sodium salt of phosphovanadotungstic acid, a copper salt of phosphovanadotungstic acid, a gold salt of phosphovanadotungstic acid and a gallium salt of phosphovanadotungstic acid.

The acid catalyst can be used as it is but is preferably supported on a support. In this case, the acid catalyst content is preferably from 10 to 200 mass %, more preferably from 50 to 150 mass %, based on the entire mass of the support.

If the acid catalyst content is less than 10 mass %, the content of active components in the catalyst is excessively small and the activity per the catalyst unit mass may disadvantageously decrease.

If the acid catalyst content exceeds 200 mass %, the effective surface area decreases and, as a result, the effect obtainable by the increase in the supported amount may not be brought out and at the same time, coking is readily generated to greatly shorten the catalyst life.

The substance which can be used as the support for the acid catalyst of the present invention is not particularly limited and those capable of providing, when prepared as a catalyst having supported thereon the acid catalyst, a catalyst having a specific surface area, by the BET method, of 65 to 350 m²/g are preferred.

The shape of the substance which can be used as the support for the catalyst of the present invention is not particularly limited and specifically, a powder, spheres, pellets and other optional forms may be used. Specific examples of the substance as the support include silica, kieselguhr, montmorillonite, titania, activated carbon, alumina and silica alumina, however, the present invention is not limited thereto.

The support is preferably a support comprising a siliceous main component and having a spherical or pellet form. The support is preferably a silica having a purity of 85 wt % or more, more preferably 95 wt % or more, based on the entire weight of the support and at the same time, having a compression strength of 30 N or more. The “compression strength” as used herein can be measured in accordance with, for example, JIS Z 8841 “Granulated Material—Strength Test Method”.

The average diameter thereof is preferably from 2 to 10 mm in the case of a fixed bed and from powder to 5 mm in the case of a fluid bed, though this varies depending on the reaction form.

The acid catalyst for use in the present invention can be produced by a desired method. An example of the method for producing a heteropolyacid and/or heteropolyacid salt catalyst is described below.

First Step:

This is a step for obtaining a solution or suspension of a heteropolyacid and/or heteropolyacid salt.

Second Step:

This is a step for loading the solution or suspension obtained in the first step on a support.

The solvent which can be used in the first step is not particularly limited as long as it can uniformly dissolve or suspend the desired heteropolyacid and/or heteropolyacid salt, and for example, water, an organic solvent or a mixture thereof may be used. Preferred examples of the solvent include water, alcohols and lower aliphatic carboxylic acids, however, the present invention is not limited thereto.

The method for dissolving or suspending a heteropolyacid and/or a heteropolyacid salt in the solvent is not particularly limited and any method may be used as long as it can uniformly dissolve or suspend the desired heteropolyacid and/or heteropolyacid salt.

For example, in the case of a heteropolyacid, namely, in the state of a free acid, when it can dissolve, the heteropolyacid may be dissolved as it is. Even when the heteropolyacid cannot be completely dissolved, if the heteropolyacid can be uniformly suspended by forming it into fine powder, the heteropolyacid may be suspended as such. In the case of a heteropolyacid salt, a method of dissolving simultaneously or separately a heteropolyacid and a starting material salt of a neutralization element and then mixing them to prepare a uniform solution or suspension may be used. In the case of a compound in the state of a heteropolyacid salt, a uniform solution or suspension may be obtained in the same manner as in the case of a heteropolyacid.

The optimal volume of the solution or suspension varies depending on the loading method in the second step and the support used but this is not particularly limited.

The second step is a step for loading a solution or suspension of a heteropolyacid and/or a heteropolyacid salt obtained in the first step on a support to obtain a catalyst for use in the production of a lower aliphatic carboxylic acid ester.

The method for loading the solution or suspension of a heteropolyacid and/or a heteropolyacid salt on a support is not particularly limited and a known method may be used.

For example, the catalyst may be prepared by dissolving or suspending a heteropolyacid and/or a heteropolyacid salt in a solvent to obtain a solution or suspension corresponding to the liquid absorption amount of a support and impregnating the solution or suspension into the support.

The catalyst may also be prepared by using an excess solution or suspension, impregnating it into a support while appropriately moving the support in the heteropolyacid solution and then removing the excess acid through filtration.

In the case of loading a heteropolyacid salt, a method of loading a heteropolyacid and at the same time, forming it into a salt using an element contained in the support and capable of forming a salt may also be used, in addition to the above-described method of previously preparing a heteropolyacid salt and then loading it.

The thus-obtained wet catalyst is preferably dried by placing it in a heating oven for a few hours. Thereafter, the catalyst is cooled to the ambient temperature in a desiccator. If the drying temperature exceeds about 400° C., the skeleton of the heteropolyacid is disadvantageously destructed. The drying temperature is preferably from 80 to 350° C.

Industrially, the catalyst may be continuously dried using a dryer such as through-flow rotary dryer, continuous fluidized bed dryer or continuous hot air carrier type dryer.

The amount of the heteropolyacid supported can be calculated simply by subtracting the weight of the support used from the dry weight of the catalyst prepared. A more exact amount can be measured by chemical analysis such as ICP (induction coupled plasma emission spectrometry).

In practicing the production process of a lower aliphatic carboxylic acid ester of the present invention, the ratio between the lower olefin and the lower aliphatic carboxylic acid used is preferably such that the lower olefin is used in an equimolar amount or excess molar amount to the lower aliphatic carboxylic acid. The ratio of lower olefin:lower aliphatic carboxylic acid is preferably, as a molar ratio, from 1:1 to 30:1, more preferably from 3:1 to 20:1, still more preferably from 5:1 to 15:1.

In the production process of a lower aliphatic carboxylic acid ester of the present invention, the vapor phase reaction may be performed in either a fixed bed form or a fluidized bed form. The shape of the support may also be selected from those formed into a size from powder to a few mm in particle size according to the form in practicing the process.

In the production process of a lower aliphatic carboxylic acid ester of the present invention, it is preferred, in view of the catalyst life, to mix a slight amount of water in the starting materials. However, if an excessively large amount of water is added, by-products such as alcohol and ether disadvantageously increase. In general, the amount of water is preferably from 1 to 15 mol %, more preferably from 2 to 8 mol %, based on the entire amount of the olefin and lower aliphatic carboxylic acid used.

The reaction temperature and the reaction pressure must be in the range of keeping the gaseous form of supply medium and vary depending on the starting materials used. In general, the reaction temperature is preferably from 120 to 250° C., more preferably from 140 to 220° C.

The pressure is preferably from atmospheric pressure to 3 MPa, more preferably from atmospheric pressure to 2 MPa.

With respect to the space velocity (hereinafter referred to as “GHSV”) of the starting materials fed to the catalyst, these are preferably passed through the catalyst layer at a GHSV of 100 to 7,000/hr, more preferably from 300 to 3,000/hr.

The present invention is described in greater detail below by referring to the Examples and the Reference Examples, however, these Examples are only for describing the outline of the present invention, and the present invention should not be construed as being limited thereto.

Analysis of Reaction Gas

With respect to the acetylene concentration in Example 1, a part of the ethylene was sampled and analyzed under the gas chromatography conditions described later. The detection limit in the analysis conditions was 1 ppm.

With respect to the acetylene concentration at the inlet of the reaction tube in Example 2 and Comparative Examples 1 and 2, an ethylene containing 0.1 vol % of acetylene was added in place of a part of ethylene which was used in Example 1 where acetylene was not detected, and a part of ethylene after the addition was sampled and analyzed by gas chromatography.

In the analysis of a gas at the outlet of the reaction tube, the whole amount of the gas was cooled and the whole amount of the condensed reaction solution collected was recovered and analyzed by gas chromatography. As for the effluent gas remaining uncondensed, the flow rate of the outlet gas flowing out within the sampling time was measured, a part of the gas was sampled and the composition was analyzed by gas chromatography. The analysis conditions are shown below.

Conditions for Analysis of Uncondensed Gas

In the analysis, an absolute calibration curve method was used and 50 ml of the effluent gas was sampled and entirely passed to a 1 ml-volume gas sampler attached to the gas chromatograph. The analysis was performed under the following conditions.

1. Ether, Lower Aliphatic Carboxylic Acid, Lower Aliphatic Carboxylic Acid Ester, Alcohol, Trace By-Products

Gas Chromatography:
gas chromatograph (GC-14B, manufactured by Shimadzu Corporation) with a gas sampler (MGS-4, measuring tube: 1 ml) for Shimadzu gas chromatograph

Column:
packed column SPAN80 15% Shinchrom A, 60 to 80 mesh (length: 5 m)

Carrier Gas:
nitrogen (flow rate: 25 ml/min)

Temperature Conditions:
constant temperature conditions that the detector and the vaporization chamber were at 120° C. and the column was at 65° C.

Detector:
FID (H₂ pressure: 60 kPa, air pressure: 100 kPa)

2. Acetylene

Gas Chromatography:
gas chromatograph (GC-14B, manufactured by Shimadzu Corporation) with a gas sampler (MGS-4, measuring tube: 2.5 ml) for Shimadzu gas chromatograph

Column:
packed column Carbosieve G, 60 to 80 mesh, length: 1 m

Carrier Gas:
N₂ (flow rate: 44 ml/min)

Temperature Conditions:
constant temperature conditions that the detector and the vaporization chamber were at 150° C. and the column was at 100° C.

Detector:
FID (H₂ pressure: 60 kPa, air pressure: 70 kPa)

3. Lower Olefin

Gas Chromatography:
gas chromatograph (GC-14B, manufactured by Shimadzu Corporation) with a gas sampler (MGS-4, measuring tube: 1 ml) for Shimadzu gas chromatograph

Column:
packed column Unibeads IS, length: 3 m

Carrier Gas:
helium (flow rate: 20 ml/min)

Temperature Conditions:
constant temperature conditions that the detector and the vaporization chamber were at 120° C. and the column was at 65° C.

Detector:
TCD (He pressure: 70 kPa, current: 90 mA, temperature: 120° C.)

Analysis of Collected Solution

The analysis was performed using the internal standard method, where the analysis solution was prepared by adding 1 ml of 1,4-dioxane as the internal standard to 10 ml of the reaction solution and 0.2 μl of the analysis solution was injected.

Gas Chromatography:
GC-14B, manufactured by Shimadzu Corporation

Column:
capillary column TC-WAX (length: 30 m, internal diameter: 0.25 mm, film thickness: 0.25 μm)

Carrier Gas:
nitrogen (split ratio: 20, column flow rate: 2 ml/min)

Temperature Conditions:
the detector and the vaporization chamber were at 200° C. and the column was kept at 50° C. for 5 minutes from the initiation of analysis and, thereafter, elevated up to 150° C. at a temperature rising rate of 20° C./min and kept at 150° C. for 10 minutes

Detector:
FID (H₂ pressure: 70 kPa, air pressure: 100 kPa)

Support

Synthetic silica (CARiACT Q-10, produced by Fuji Silysia Chemical Ltd.) (specific surface area: 219.8 m²/g, pore volume: 0.660 cm³/g) was used.

Preparation Method of Catalyst

The support was dried for 4 hours in a (hot air) dryer adjusted to 110° C. Silicotungstic acid and lithium nitrate were weighed to 34.99 g and 0.0837 g, respectively, 15 ml of pure water was added thereto and the mixture was uniformly dissolved to obtain an aqueous Li_{0.1}H_{2.9}PW₁₂O₄₀ solution (impregnating solution). To the impregnating solution, 100 ml of the support was added and thoroughly stirred. The support impregnated with the solution was air dried for 1 hour and thereafter dried for 5 hours by a dryer adjusted to 150° C. In the catalyst obtained, the supported amount was 300 g/liter.

### EXAMPLE 1

After filling 40 ml of the catalyst into a reaction tube, a reaction was continuously performed for 400 hours by passing a starting material gas consisting of ethylene:acetic acid:water vapor:nitrogen at a volume ratio of 78.5:8.0:4.5:9.0 and prepared using a high-purity ethylene containing no acetylene, through the reaction tube at a rate of 80.77 g/hour under a pressure of 0.8 MPaG while keeping the highest temperature portion of the catalyst layer at 165° C. The results are shown in Table 1.

TABLE 1

| | Acetylene | | | |
|---|---|---|---|---|
| | Concentration | | STY of | Activity |
| | at the Inlet | Reaction | Ethyl | Reduction |
| | of a Reactor | Time | Acetate | Rate (STY |
| | (ppm) | (hr) | (g/L-hr) | drop/100 hr) |
| Example 1 | not detected | 5 | 243.0 | 0.3 |
| | | 403 | 241.8 | |
| Example 2 | 25 | 5 | 245.1 | 1.8 |
| | | 408 | 237.8 | |
| Comparative | 51 | 5 | 241.3 | 2.3 |
| Example 1 | | 410 | 232.0 | |
| Comparative | 103 | 5 | 246.5 | 5.2 |
| Example 2 | | 400 | 226.0 | |

* The acetylene concentration corresponds to the molar ratio of acetylene to the total of acetylene and ethylene.

### EXAMPLE 2

A reaction was performed in the same manner as in Example 1 except for using an acetylene-containing ethylene in place of a part of the high-purity ethylene and adjusting the acetylene concentration in the starting material gas to 25 ppm based on the total of acetylene and ethylene. The results are shown in Table 1.

### COMPARATIVE EXAMPLE 1

A reaction was performed in the same manner as in Example 1 except for using an acetylene-containing ethylene in place of a part of the high-purity ethylene and adjusting the acetylene concentration in the starting material gas to 51 ppm based on the total of acetylene and ethylene. The results are shown in Table 1.

### COMPARATIVE EXAMPLE 2

A reaction was performed in the same manner as in Example 1 except for using an acetylene-containing ethylene in place of a part of the high-purity ethylene and adjusting the acetylene concentration in the starting material gas to 103 ppm based on the total of acetylene and ethylene. The results are shown in Table 1.

### INDUSTRIAL APPLICABILITY

As is apparent from the results shown above, in the process for producing a lower aliphatic carboxylic acid ester from a lower aliphatic carboxylic acid and a lower olefin in the presence of an acid catalyst, a stable operation can be continuously performed for a long period of time by controlling the starting materials to contain substantially no acetylenes.

1. A process for producing a lower aliphatic carboxylic acid ester from a lower aliphatic carboxylic acid and a lower olefin in the presence of an acid catalyst, wherein the starting materials contain substantially no acetylene compounds.

2. The process as set forth in claim 1, wherein the concentration of acetylene compounds is 25 ppm or less in terms of the molar ratio to the total of the acetylene compounds and the lower olefin.

3. The process as set forth in claim 1, wherein the concentration of acetylene compounds is 10 ppm or less in terms of the molar ratio to the total of the acetylene compounds and the lower olefin.

4. The process as set forth in claim 1, wherein the concentration of acetylene compounds is 1 ppm or less in terms of the molar ratio to the total of the acetylene compounds and the lower olefin.

5. The process as set forth in any one of claims 1 to 4, wherein the acetylene compound is acetylene.

6. The process as set forth in any one of claims 1 to 4, wherein the acetylene compound is methyl acetylene.

7. The process as set forth in any one of claims 1 to 4, wherein the acetylene compound is vinyl acetylene.

8. The process as set forth in any one of claims 1 to 7, wherein the lower aliphatic carboxylic acid is at least one lower aliphatic carboxylic acid having from 1 to 4 carbon atoms.

9. The process as set forth in any one of claims 1 to 8, wherein the lower olefin is at least one olefin selected from the group consisting of ethylene, propylene, n-butene, isobutene and a mixture of two or more thereof.

10. The process as set forth in any one of claims 1 to 9, wherein the acid catalyst contains at least one compound selected from heteropolyacids and heteropolyacid salts.

11. The process as set forth in claim 10, wherein the heteropolyacids are selected from the group consisting of a silicotungstic acid, a phosphotungstic acid, a phosphomolybdic acid, a silicomolybdic acid, a silicovanadotungstic acid, a phosphovanadotungstic acid and a phosphovanadomolybdic acid.

12. The process as set forth in claim 10, wherein the heteropolyacid salts are selected from the group consisting of lithium salts, sodium salts, potassium salts, cesium salts, magnesium salts, barium salts, copper salts, gold salts, gallium salts and ammonium salts of silicotungstic acid, phosphotungstic acid, phosphomolybdic acid, silicomolybdic acid, silicovanadotungstic acid, phosphovanadotungstic acid and phosphovanadomolybdic acid.

13. The process as set forth in any one of claims 1 to 12, wherein the reaction between the lower olefin and the lower aliphatic carboxylic acid is performed in the presence of water.

14. A lower aliphatic carboxylic acid ester produced by the process according to in any one of claims 1 to 13.
